(19) United States
(12) Patent Application Publication
(10) Pub. No.: US 2008/0301859 A1
(43) Pub. Date: Dec. 11, 2008

(54) Bridge Device for Swimming Goggles

(76) Inventor: Terry Chou, Tainan City (TW)

Correspondence Address: Dr. BANGER SHIA;Int''l Patent Office of Bang Shia, 102 Lindencrest Ct, Sugar Land, TX, 77479-5201, US

(21) Appl. No.: 11/759,924

(22) Filed: Jun. 7, 2007

(51) Int. Cl. A61F 9/02

(52) U.S. Cl. 2/445; 2/426

(57) ABSTRACT

A pair of swimming goggles includes two bodies each including an inner side having an upper coupling section on an upper portion thereof and a lower coupling section on a lower portion thereof. A first bridging member includes a first coupling portion on each of two ends thereof. The first coupling portions of the first bridging member are respectively and releasably coupled with two of the upper and lower coupling sections of the bodies. A second bridging member includes a second coupling portion on each of two ends thereof. The second coupling sections of the second bridging member are respectively and releasably coupled with the other two of the upper and lower coupling sections of the bodies. A fixed spacing between the bodies is provided by the first and second bridging members.

### BACKGROUND OF THE INVENTION

1. Field of the Invention

The present invention relates to a bridge device for a pair of goggles and, more particularly, to a bridge device for a pair of swimming goggles.

2. Description of the Related Art

A typical pair swimming goggles includes two bodies, a bridge between the bodies, and a head strap. Each body includes a lens coupled to a frame or a padding member while allowing the bodies to be in intimate contact with the eye sockets of a user.

FIG. 9 of the drawings illustrates a pair of conventional swimming goggles including two bodies 1′ and 2′, a bridge 3′, and a head strap 4′. Each body 1′, 2′ includes a lens 11′, 21′ and a padding member 12′, 22′. Each lens 11′, 21′ includes an inner coupling portion 111′, 211′ with an opening 112′, 212′ for coupling with the bridge 3′. The bridge 3′ is made of a flexible material and includes a plurality of ridges 31′ in an inner face thereof. The bridge 3′ is substantially U-shaped and has two distal ends respectively extending through the openings 112′ and 212′ for connecting the bodies 1′ and 2′ together. However, the bodies 1′ and 2′ are liable to shift relative to each other (see FIG. 10), failing to provide a reliable assembly.

FIG. 11 illustrates another pair of conventional swimming goggles including two bodies 5′ and 6′, a bridge 7′, and a head strap 8′. Each body 5′, 6′ includes a soft frame 51′, 61′ with an integrally formed padding member 52′ 62′ on an inner face thereof for intimate contact with an eye socket of a user. Since the bridge 7′ is integrally formed between the soft frames 51′ and 61′ made of a material the same as the bridge 7′, the problem of relative shift between the bodies 5′ and 6′ still exists. Furthermore, the bridge 7′ cannot be adjusted in length; namely, the distance between the bodies 5′ and 6′ is not adjustable and, thus, cannot provide flexible applications.

FIG. 12 illustrates a pair of swimming goggles disclosed in U.S. Patent Publication No. 2007/0022521 A1. Specifically, the pair of swimming goggles includes two ocular assemblies 91′ and 92′ each having a stud 911′, 912′ at a lower end of an inner portion thereof and a shoulder with a hole 912′, 922′ at an upper end of the inner portion thereof for the purposes of engaging with a bridging member 93′. The bridging member 93′ includes an upper stalk and a lower stalk. Each lower stalk has an open hole 931′ for engaging the stud 911′, 912′. Each upper stalk is extended through the hole 912′, 922′ and has a succession of ridges 932′ on the end thereof. However, the ridges 932′ on the upper stalks are liable to disengage from the holes 912′ and 922′.

U.S. Pat. No. 5,502,844 discloses a pair of swimming goggles including two eye pieces each having a lens. Each eye piece has a plurality of holes in an inner edge thereof. A pull cord is extended through some of the holes whereas another pull cord is extended through the other holes to provide a bridge between the eye pieces. However, the eye pieces are liable to shift relative to each other while failing to provide a reliable assembly.

It is therefore a need in a bridge device for a pair of swimming goggles providing enhanced assembling reliability and allowing flexible applications.

### SUMMARY OF THE INVENTION

A pair of swimming goggles according to the preferred teachings of the present invention includes two bodies each including an inner side having an upper coupling section on an upper portion thereof and a lower coupling section on a lower portion thereof. A first bridging member includes a first coupling portion on each of two ends thereof. The first coupling portions of the first bridging member are respectively and releasably coupled with two of the upper and lower coupling sections of the bodies. A second bridging member includes a second coupling portion on each of two ends thereof. The second coupling sections of the second bridging member are respectively and releasably coupled with the other two of the upper and lower coupling sections of the bodies. A fixed spacing between the bodies is provided by the first and second bridging members.

In an embodiment, the first coupling portions of the first bridging member are respectively coupled with the upper coupling sections of the bodies, and the second coupling portions of the second bridging member are respectively coupled with the lower coupling sections of the bodies. The second bridging member is below and spaced from the first bridging member. The second bridging member includes a bend extending upward between the ends thereof for preventing a users nose from being pressed.

In another embodiment, the first coupling portions of the first bridging member are respectively coupled with the upper coupling section of one of the bodies and the lower coupling section of the other body whereas the second coupling portions of the second bridging member are respectively coupled with the remaining coupling sections of the bodies. The first and second bridging members extend across each other. A space below an intersection of the first and second bridging members receives a user's nose.

In an embodiment, each coupling section of each body includes a stub having an enlarged distal end. Each of the first and second coupling portions of the first and second bridging members includes a through-hole having a diameter slightly smaller than that of the enlarged distal end of the stub. The stub of each coupling section of each body is engaged with the through-hole of an associated one of the first and second coupling portions.

In another embodiment, each coupling section of each body includes a stub having an enlarged distal end. Each of the first and second coupling portions of the first and second bridging members includes a plurality of through-holes having a diameter slightly smaller than that of the enlarged distal end of the stub. The stub of each coupling section of each body is selectively engaged with one of the plurality of through-holes of an associated one of the first and second coupling portions.

In a further embodiment, each of the first and second coupling portions of the first and second bridging members includes a stub. Each coupling section of each body includes a hole. The stub of each of the first and second coupling portions of the first and second bridging members is engaged with the through-hole of an associated one of the upper and lower coupling sections of the bodies.

Other objectives, advantages, and novel features of the invention will become more apparent from the following detailed description when taken in conjunction with the accompanying drawings.

### BRIEF DESCRIPTION OF THE DRAWINGS

FIG. 1 shows a partially exploded perspective view of a first embodiment of a pair of swimming goggles according to the preferred teachings of the present invention.

FIG. 2 shows a perspective view of the pair of swimming goggles of FIG. 1.

FIG. 3 shows a front elevational view of the pair of swimming goggles of FIG. 2.

FIG. 4 shows a front elevational view of a second embodiment of a pair of swimming goggles according to the preferred teachings of the present invention.

FIG. 5 shows a front elevational view of a third embodiment of a pair of swimming goggles according to the preferred teachings of the present invention.

FIG. 6 shows a front elevational view of a fourth embodiment of a pair of swimming goggles according to the preferred teachings of the present invention.

FIG. 7 shows a partially exploded perspective of a fifth embodiment of a pair of swimming goggles according to the preferred teachings of the present invention.

FIG. 8 shows a perspective view of the pair of swimming goggles of FIG. 7.

FIG. 9 shows a perspective view, partly exploded, of a pair of conventional swimming goggles.

FIG. 10 shows a front elevational view of the pair of swimming goggles of FIG. 9.

FIG. 11 shows a perspective view of another pair of conventional swimming goggles.

FIG. 12 shows a front elevational view of a pair of swimming goggles disclosed in U.S. Patent Publication No. 2007/0022521 A1.

### DETAILED DESCRIPTION OF THE PREFERRED EMBODIMENTS

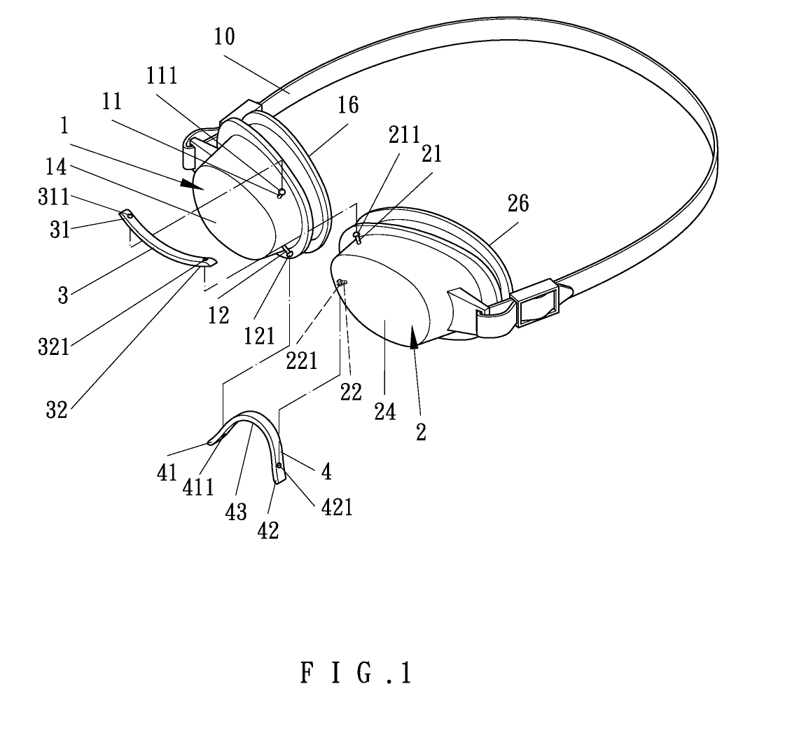

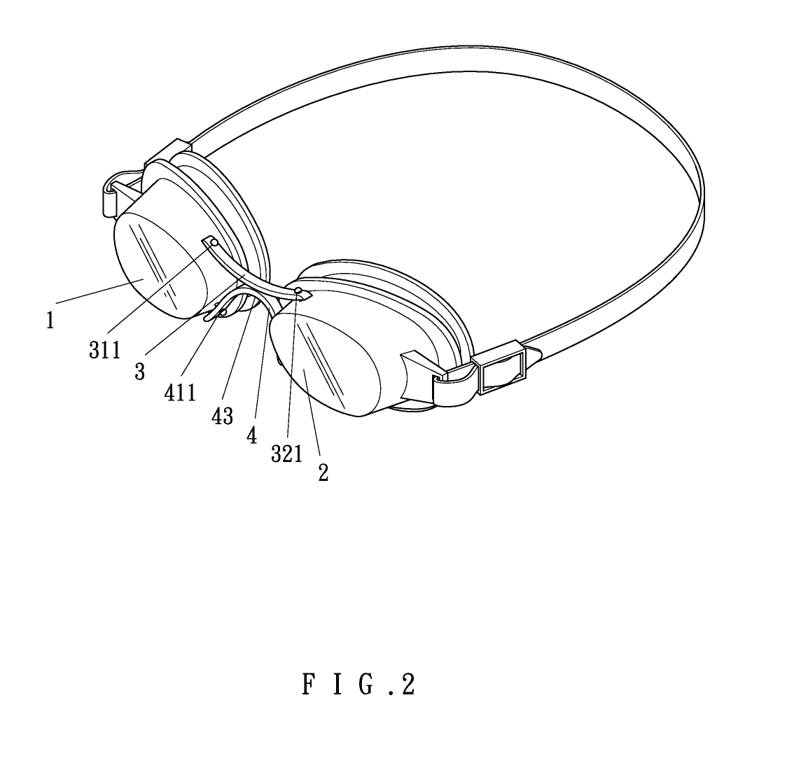

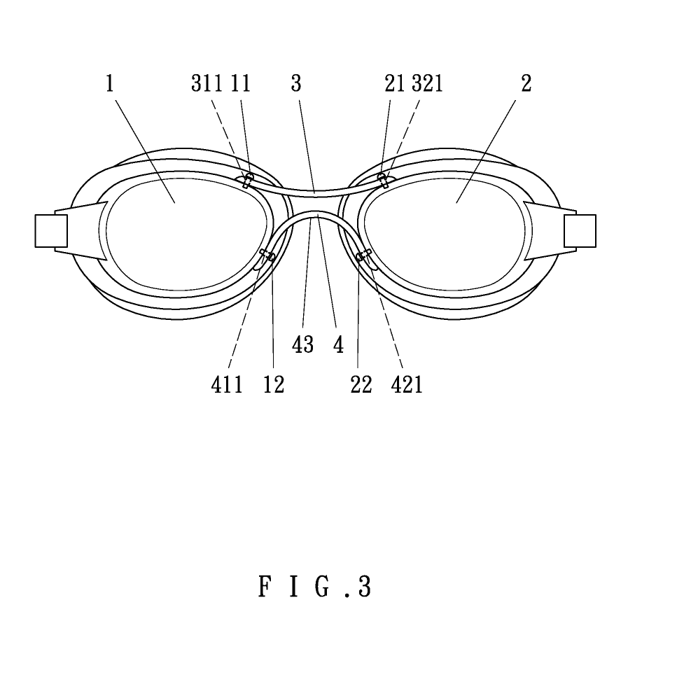

Referring to FIGS. 1 through 3, a first embodiment of a pair of swimming goggles according to the preferred teachings of the present invention includes two bodies 1 and 2, a bridge device, and a head strap 10. In the preferred form shown, the bridge device includes a first bridging member 3 and a second bridging member 4 that are interconnected between the bodies 1 and 2. Each body 1, 2 includes a rigid lens 14, 24 and a soft padding member 16, 26 coupled to the rigid lens 14, 24. Each lens 14, 24 includes an upper coupling section 11, 21 on an upper portion of an inner side thereof and a lower coupling section 12, 22 on a lower portion of the inner side thereof. Each coupling section 11, 12, 21, 22 is in the form of a stub having an enlarged distal end 111, 121, 211, 221.

Each of the first and second bridging members 3, 4 is made of a rigid material and has an appropriate length. Each of the first and second bridging members 3, 4 includes a coupling portion 31, 32, 41, 42 on each of two ends thereof. In the illustrated example, each coupling portion 31, 32, 41, 42 has a through-hole 311, 321, 411, 421 having a diameter slightly smaller than the enlarged distal end 111, 121, 211, 221 of the coupling section 11, 12, 21, 22. Furthermore, the lower bridging member 4 includes a bend 43 extending upward between two ends thereof.

In assembly, the through-holes 311 and 321 of the ends of the first bridging member 3 are coupled with the upper coupling sections 11 and 21. Thus, the first bridging member 3 is interconnected between the bodies 1 and 2. The through-holes 411 and 421 of the ends of the second bridging member 4 are coupled with the lower coupling sections 12 and 22. Thus, the second bridging member 4 is interconnected between the bodies 1 and 2 below and spaced from the upper bridging member 3. The first and second bridging members 3 and 4 provide a fixed spacing between the bodies 1 and 2. Undesired shift between the bodies 1 and 2 is prevented by the first and second bridging members 3 and 4 that simultaneously support the bodies 1 and 2, providing enhanced assembling reliability. Meanwhile, the bend 43 of the second bridging member 4 prevents a user's nose from being pressed against by the second bridging member 4.

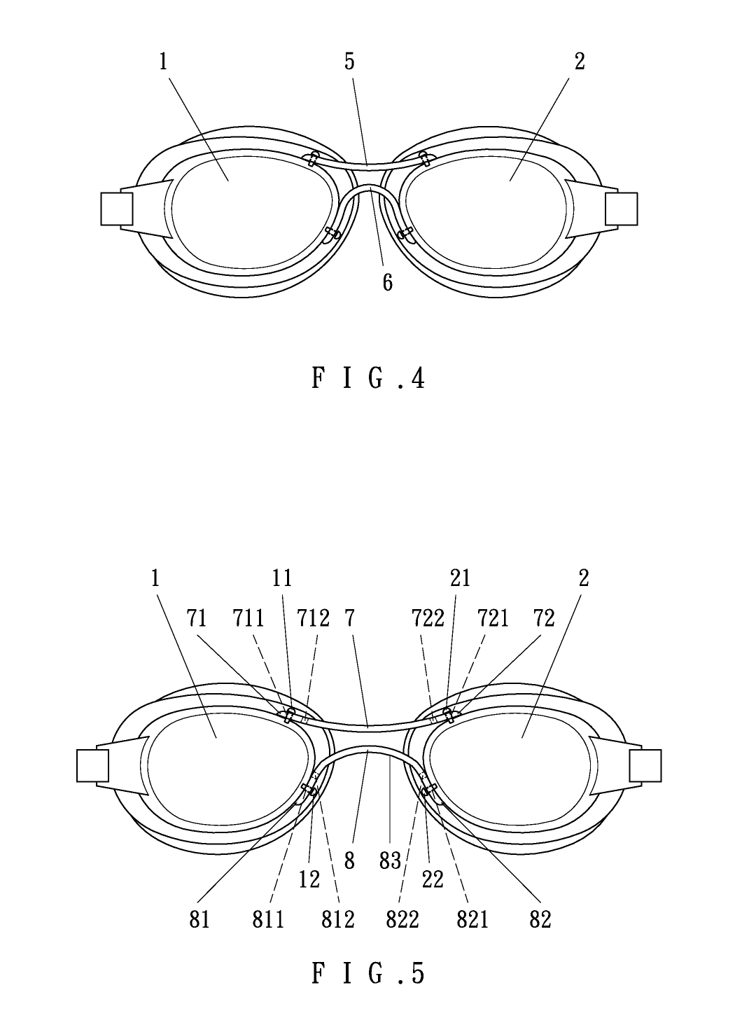

The lengths of the first and second bridging members 3 and 4 may be varied to increase or decrease the spacing between the bodies 1 and 2 for various users. Furthermore, the first and second bridging members 3 and 4 may have various colors to present an aesthetically pleasing appearance. In a second embodiment shown in FIG. 4, the first and second bridging members 3 and 4 are shorter than those in the first embodiment shown in FIGS. 1-3.

In a third embodiment shown in FIG. 5, the first bridging member (now designated 7) includes a coupling portion 71, 72, 81, 82 on each of two ends thereof. Each coupling portion 71, 72, 81, 82 has a plurality of through-holes 711 and 712, 721 and 722, 811 and 812, 821 and 822 having a diameter slightly smaller than the enlarged distal end 111, 121, 211, 221 of the coupling section 11, 12, 21, 22. Furthermore, the lower bridging member (now designated 8) includes a bend 83 extending upward between two ends thereof. By selectively engaging one of the through-holes 711, 712, 721, 722, 811, 812, 821, and 822 of the first and second bridging members 7 and 8 with the upper and lower coupling sections 11, 12, 21, and 22 the spacing between the bodies 1 and 2 can be adjusted to provide flexible application.

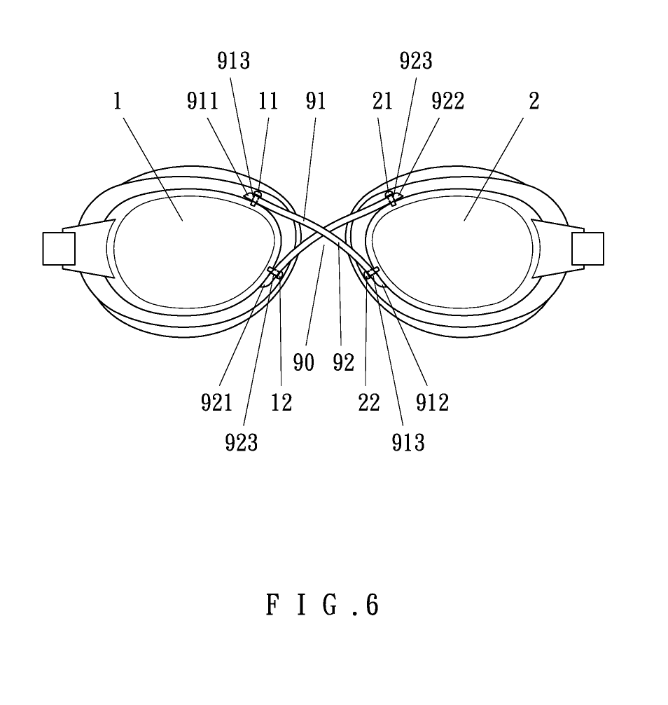

In a fourth embodiment shown in FIG. 6, the bridge device includes first and second bridging members 91 and 92 that extend across each other. The first bridging member 91 includes two coupling portions 911, 912 respectively on two ends thereof. Each coupling portion 911, 912 includes a through-hole 913 for engaging with the coupling sections 11 and 22 of the bodies 1 and 2. The second bridging member 92 includes two coupling portions 921, 922 respectively on two ends thereof. Each coupling portion 921, 922 includes a through-hole 923 for engaging with the coupling sections 12 and 21 of the bodies 1 and 2. The crosswise arranged bridging members 91 and 92 provide reliable support and a fixed spacing between the bodies 1 and 2 while preventing undesired shift of the bodies 1 and 2 in the horizontal position. Furthermore, the space 90 below an intersection (not labeled) of the first and second bridging members 91 and 92 receives the users nose and, thus, provides the user with wearing comfort.

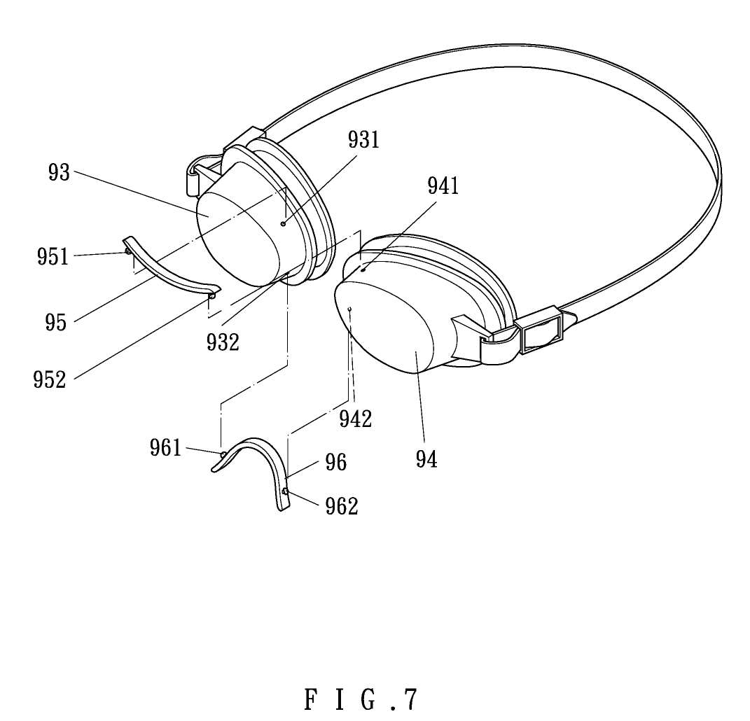

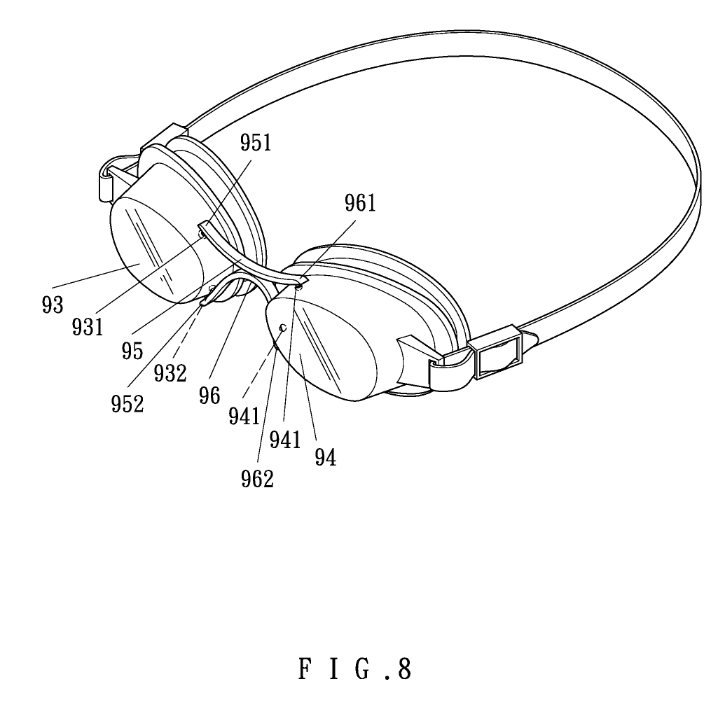

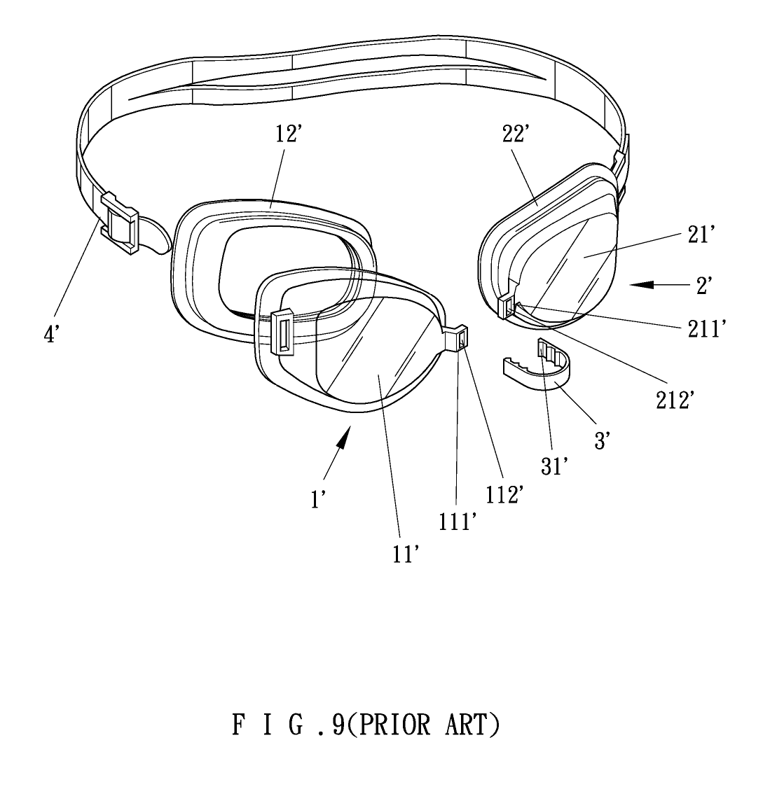

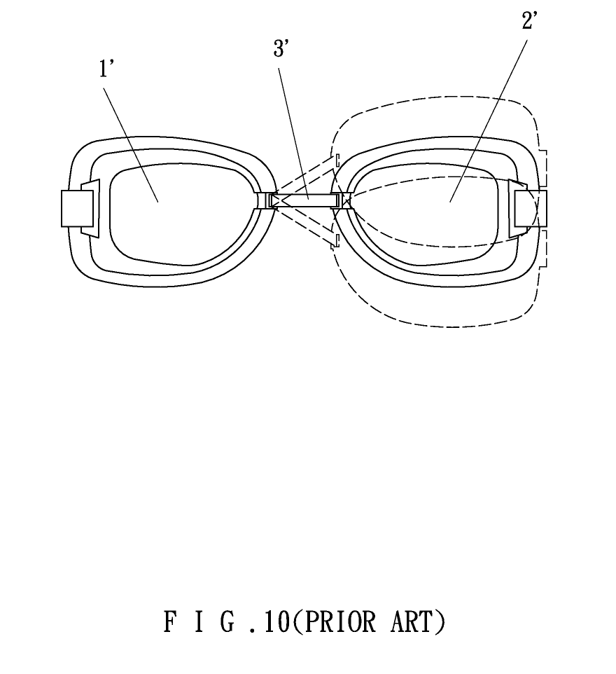

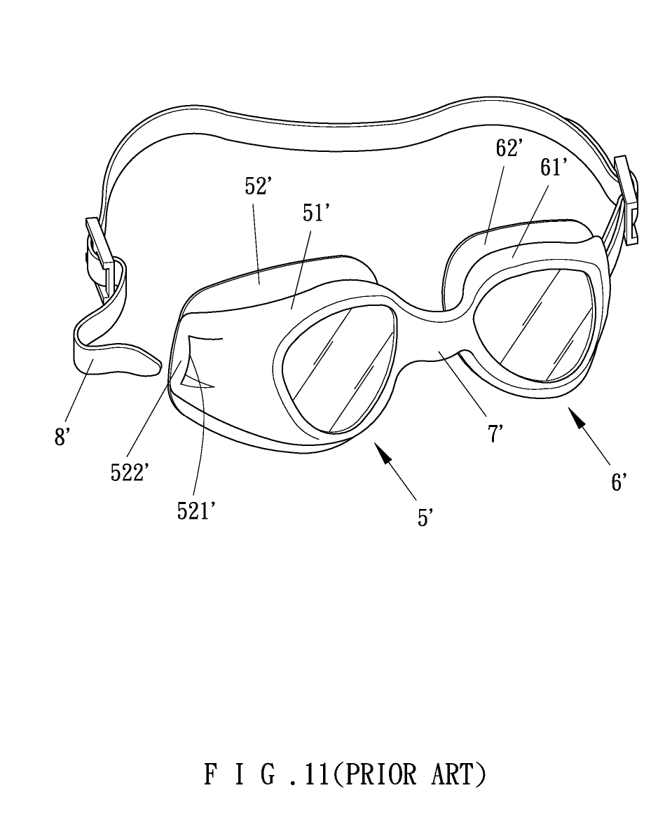

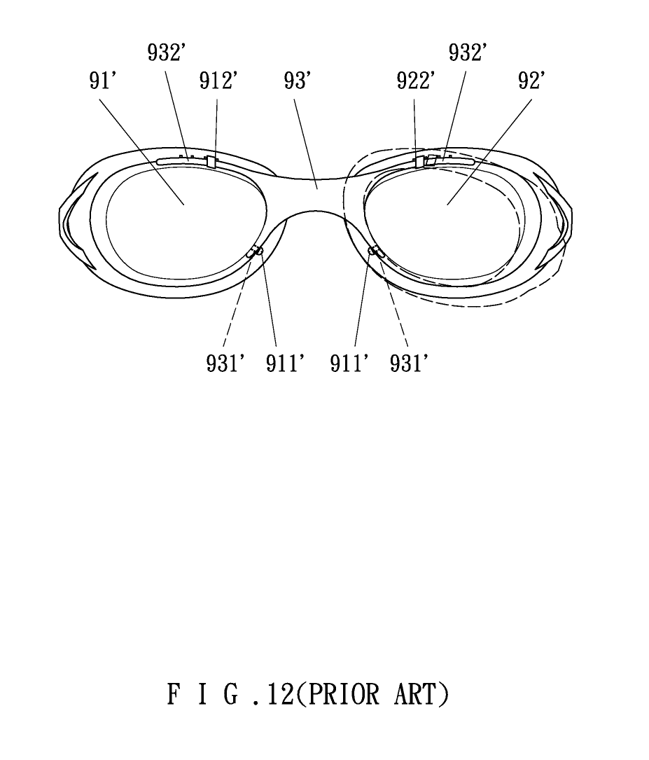

In a fifth embodiment shown in FIGS. 7-8, the pair of swimming goggles includes two bodies 93 and 94 each having a coupling section 931, 932, 941, 942 in upper and lower portions of an inner side thereof. Each coupling section 931, 932, 941, 942 has a hole (not labeled). The pair of swimming goggles further includes a bridging device having first and second bridging members 95 and 96 each having a coupling portion 951, 952, 961, 962 on each of two ends thereof. Each coupling portion 951, 952, 961, 962 is in the form of a stub for coupling with the hole of an associated coupling section 931, 932, 941, 942.

It can be appreciated that the coupling sections of the bodies and the coupling portions of the bridging members can be jointed together by screwing, gluing, or other suitable methods.

Although specific embodiments have been illustrated and described, numerous modifications and variations are still possible without departing from the teachings of the invention. The scope of the invention is limited by the accompanying claims.

What is claimed is:

1. A pair of swimming goggles comprising:
two bodies each including an inner side having an upper coupling section on an upper portion thereof and a lower coupling section on a lower portion thereof;
a first bridging member including a first coupling portion on each of two ends thereof, with the first coupling portions of the first bridging member being respectively and releasably coupled with two of the upper and lower coupling sections of the bodies; and
a second bridging member including a second coupling portion on each of two ends thereof, with the second coupling sections of the second bridging member being respectively and releasably coupled with the other two of the upper and lower coupling sections of the bodies, providing a fixed spacing between the bodies.

2. The pair of swimming goggles as claimed in claim 1, with the first coupling portions of the first bridging member being respectively coupled with the upper coupling sections of the bodies, with the second coupling portions of the second bridging member being respectively coupled with the lower coupling sections of the bodies, with the second bridging member being below and spaced from the first bridging member, and with the second bridging member including a bend extending upward between the ends thereof for preventing a user's nose from being pressed.

3. The pair of swimming goggles as claimed in claim 1, with the first coupling portions of the first bridging member being respectively coupled with the upper coupling section of one of the bodies and the lower coupling section of the other body, with the second coupling portions of the second bridging member being respectively coupled with the lower coupling section of said one of the bodies and the upper coupling section of the other body, with the first and second bridging members extending across each other, and with a space below an intersection of the first and second bridging members receiving a users nose.

4. The pair of swimming goggles as claimed in claim 1, with each of the upper and lower coupling sections of the bodies including a stub having an enlarged distal end, with each of the first and second coupling portions of the first and second bridging members including a through-hole having a diameter slightly smaller than that of the enlarged distal end of the stub, and with the stub of each of the upper and lower second coupling sections of the bodies being engaged with the through-hole of an associated one of the first and second coupling portions.

5. The pair of swimming goggles as claimed in claim 1, with each of the upper and lower coupling sections of the bodies including a stub having an enlarged distal end, with each of the first and second coupling portions of the first and second bridging members including a plurality of through-holes having a diameter slightly smaller than that of the enlarged distal end of the stub, and with the stub of each of the upper and lower coupling sections of the bodies being selectively engaged with one of the plurality of through-holes of an associated one of the first and second coupling portions.

6. The pair of swimming goggles as claimed in claim 1, with each of the first and second coupling portions of the first and second bridging members including a stub, with each of the upper and lower coupling sections of the bodies including a hole, with the stub of each of the first and second coupling portions of the first and second bridging members being engaged with the through-hole of an associated one of the upper and lower coupling sections of the bodies.
